(19) United States
(12) Patent Application Publication
(10) Pub. No.: US 2002/0055732 A1
(43) Pub. Date: May 9, 2002

(54) Catheter assembly and method for positioning the same at a bifurcated vessel

(76) Inventor: W. Stan Wilson, Missoula, MT (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/021,914

(22) Filed: Dec. 12, 2001

Related U.S. Application Data
(62) Division of application No. 09/465,101, filed on Dec. 16, 1999.

(51) Int. Cl. A61M 25/01; A61M 25/02; A61M 25/04; A61M 25/06; A61M 25/08; A61M 25/082; A61M 25/085; A61M 25/088; A61M 25/09; A61M 25/095; A61M 25/098

(52) U.S. Cl. 604/528

(57) ABSTRACT

An improved catheter assembly is provided for advancing a pair of guide wires to a bifurcated vessel in preparation for an interventional procedure. The delivery assembly of the present invention has the novel feature of containing two guide wire lumens in a single catheter to prevent wire wrapping and crossing of the wires. The tracking guide wire lumen and the integrated guide wire lumen run substantially parallel to each other throughout their lengths, and the tracking guide wire lumen and the integrated guide wire lumen do not move apart with respect to each other as the catheter is manipulated. The invention provides for a clip for clipping the wires at their proximal ends outside of the patient, thus helping to prevent wire wrapping, wire crossing, and confusion between wires. The assembly allows for the delivery of two wires to a bifurcation so that the bifurcation is ready for an interventional procedure, such as the implantation of a bifurcated stent.

### BACKGROUND OF THE INVENTION

The present invention relates to a guide wire deployment assembly for use at a bifurcation and, more particularly, to a delivery catheter assembly and method for the purpose of facilitating further interventional treatment, such as a percutaneous transluminal coronary angioplasty (PTCA) procedure.

In a PTCA procedure, a balloon catheter is used to dilate the lumen of a coronary artery which has become narrowed or restricted due to the accumulation of atherosclerotic plaque along the artery wall. A balloon catheter is advanced through the vasculature to the stenosis and the balloon is inflated to radially compress the atherosclerotic plaque against the inside of the artery wall. The balloon is then deflated so that the dilation catheter can be removed and blood flow resumed through the dilated artery.

Occasionally, the inflation of the balloon within the artery lumen will dissect either the stenotic plaque or the intima of the blood vessel or both. After the balloon is deflated and removed, blood can flow between the arterial wall and the dissected lining thereby constricting the flow passage or causing a section of the dissected lining, commonly called an “intimal flap,” to be forced into the flow passageway. In the event of partial or total occlusion of a coronary artery by a dissected arterial lining, the patient is put in an extremely dangerous situation requiring immediate medical attention, particularly when the occlusion occurs in one of the coronary arteries.

Another problem which frequently arises after an angioplasty procedure is the appearance of a restenosis at or near the site of the treated artery. The restenosis may appear due to the accumulation of additional atherosclerotic plaque or may be the result of weakened arterial walls which have collapsed inward to restrict blood flow. When restenosis appears, the treated patient may require an additional angioplasty procedure or other treatment such as by-pass surgery, if an additional angioplasty procedure is not warranted.

Due to the problems caused by dissections of the arterial lining or the appearance of restenosis, much research has been performed on ways to maintain the patency of an artery after the angioplasty procedure is completed. In recent years, expandable endoprosthetic devices, commonly called “stents,” have gained widespread acceptance as a means to support the arterial walls and maintain the patency of a treated vessel. Stents are generally cylindrically shaped intravascular devices which are placed within a damaged artery to hold it open and maintain unimpeded blood flow. Stents prevent dissected arterial linings from occluding an artery by pressing the dissected tissue against the arterial wall until natural healing results in the re-securing of the dissected tissue to the arterial wall. Stents also prevent the appearance of restenosis in the treated vessel by supporting the weakened arterial walls.

Various means have been developed for delivering and implanting intravascular stents within a body lumen. One common method involves compressing or otherwise reducing the diameter of a self-expanding stent, mounting the compressed stent on the distal end of a delivery catheter, placing a tubular sheath over the stent to restrain the stent in the contracted condition, and advancing the catheter through the patient's vasculature to the desired location. Once the stent is properly positioned, the stent is exposed by withdrawing the sheath proximally with respect to the stent, advancing the stent distally with respect to the sheath, or performing a combination of both. Once free from the sheath, the self-expanding stent expands against the arterial walls to thereby hold open the artery or other body lumen into which it is placed.

One of the difficulties with some interventional procedures, such as stenting procedures, involves placing two wires from outside the body to a position beyond a bifurcation for the purpose of further interventional treatment. Some prior art concepts require bringing a catheter assembly into the body over two wires. The delivery of such prior devices to the bifurcation is highly unreliable and often unsuccessful because of the wrapping of the second wire with the first at various points between its entry into the body and its arrival at the bifurcation. Wrapping can occur anywhere between entry of wires into a rotating hemostatic valve or a guide catheter and the bifurcation target. Wrapping that can occur within the guide may be pushed distally and only evident as the device in question enters the proximal coronary circulation. It is impossible to steer a second wire under fluoroscopy without crossing first in front of, then behind the first wire as the second wire reaches tortuosity. When such a prior art catheter assembly encounters a wrap, it fails to advance and the delivery of such a device is unsuccessful. The present invention solves these and other problems.

As used herein, the terms “proximal,” “proximally,” and “proximal direction” when used with respect to the invention are intended to mean moving away from or out of the patient, and the terms “distal,” “distally,” and “distal direction” when used with respect to the invention are intended to mean moving toward or into the patient. These definitions will apply with reference to apparatus, such as catheters, guide wires, stents, the like.

### SUMMARY OF THE INVENTION

The present invention provides for an improved design for a catheter assembly and method for positioning the same at a bifurcated vessel. The delivery assembly of the present invention has the novel feature of containing two guide wire lumens in a single catheter to prevent wire wrapping and crossing of the wires. Additionally, the invention provides for a clip for holding the wires at their proximal ends outside of the patient, thus helping to prevent wire wrapping, wire crossing, and confusion when identifying and manipulating the two wires.

In one aspect of the invention, there is provided a delivery assembly for treating bifurcated vessels having a main vessel and a side branch vessel, the assembly including an elongated catheter. A tracking guide wire lumen is provided for receiving a tracking guide wire. The tracking guide wire lumen extends through at least a portion of the catheter. An integrated guide wire lumen is included for receiving an integrated guide wire. The integrated guide wire lumen extends through at least a portion of the catheter. The tracking guide wire lumen and the integrated guide wire lumen run substantially parallel to each other throughout their lengths, and the tracking guide wire lumen and the integrated guide wire lumen remain substantially next to each other as the catheter is manipulated.

Additionally, the invention provides for a clip for clipping the wires at their proximal ends outside of the patient, thus helping to prevent wire wrapping, wire crossing, and confusion when identifying and manipulating the two wires. The assembly allows for the delivery of two wires to a bifurcation so that the bifurcation is ready for an interventional procedure, such as the implantation of a bifurcated stent. It is contemplated that the tracking guide wire lumen and the integrated guide wire lumen can be of the over-the-wire (OTW), rapid-exchange (RX), or unzippable-rapid-exchange types, which are known to those skilled in the art.

The present invention also is directed to a method of preparing a bifurcated vessel having a bifurcation, a main vessel, and a side branch vessel, for an interventional procedure. The method includes the step of providing an elongated catheter having a tracking guide wire lumen for receiving a tracking guide wire, the tracking guide wire lumen extending through at least a portion of the catheter. The catheter further includes an integrated guide wire and integrated guide wire lumen for receiving an integrated guide wire, the integrated guide wire lumen also extending through at least a portion of the catheter. The tracking guide wire lumen and the integrated guide wire lumen run substantially parallel to each other throughout their lengths, and the tracking guide wire lumen and the integrated guide wire lumen do not move apart with respect to each other.

A tracking guide wire can be back loaded into the tracking guide wire lumen. The catheter is then advanced over the tracking guide wire to a position proximal of the bifurcation in the main vessel. Next, the integrated guide wire is advanced through the integrated guide wire lumen and into the side vessel branch. The catheter can then be removed from the patient's vasculature. In one embodiment, a retaining element, such as a clip, is provided for retaining the tracking guide wire and the integrated guide wire. The tracking guide wire and the integrated guide wire can be retained within the retaining element by clipping the wires at their proximal ends outside of the patient. This helps to prevent wire wrapping, wire crossing, and confusion between wires.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is an elevational view of a bifurcation in which a prior art attempt is unsuccessfully made to correctly deliver two guide wires.

FIG. 2 is a perspective view of a first embodiment of a delivery assembly of the present invention.

FIG. 3 is a longitudinal cross-sectional view of the catheter of FIG. 2 depicting two guide wire lumens.

FIG. 4 is an elevational view of a bifurcated vessel in which the catheter of FIG. 2 is advanced over a tracking guide wire to a location just proximal to a side branch vessel.

FIG. 5 is an elevational view of a bifurcated vessel in which an integrated guide wire is advanced out of the catheter of FIG. 2 and into a side branch vessel.

FIG. 6 is a perspective view of a novel clip of the present invention.

FIG. 7 is a perspective view of another embodiment of a delivery assembly of the present invention.

FIG. 8 is a longitudinal cross-sectional view of the catheter of FIG. 7 depicting two guide wire lumens.

### DETAILED DESCRIPTION OF THE PREFERRED EMBODIMENTS

As shown in the exemplary drawings wherein like reference numerals indicate like or corresponding elements among the figures, the present invention includes a catheter assembly and method for positioning the same.

As mentioned above, some attempts at providing stenting solutions for bifurcations have implemented the use of two guide wires in the body at once for preparation of interventional treatment at a bifurcation wherein one guide wire is fed into a main vessel and another guide wire is fed into a side branch vessel. When the second of these wires is advanced it is impossible to visually or otherwise prevent this wire from passing first anterior to and then posterior to the first wire, particularly within tortuosity within the guide or in the proximal portion of the native vessel system. This results in wrapping of the wires. Such wrapping becomes evident when an attempt is made to bring a device or devices over these two wires to reach the point of bifurcation. When the device or devices encounter these wraps, resistance to advancement of the device occurs, often not permitting its delivery to the bifurcation.

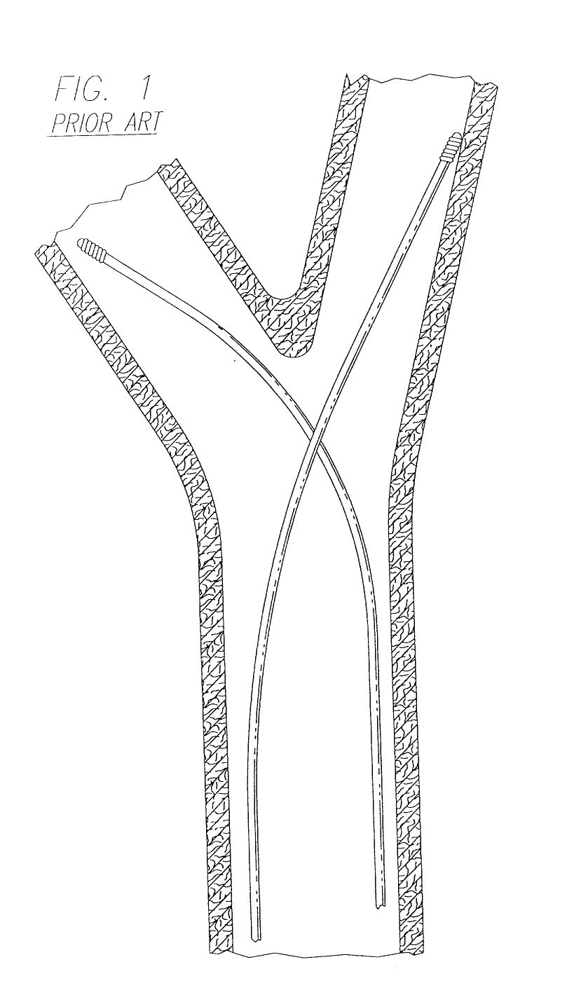

FIG. 1 depicts one such scenario that has been encountered in the past when attempting to deliver two guide wires to a bifurcation. The wires are prone to crossing, which causes problems when proceeding with further interventional treatment, such as the delivery of a stent. Furthermore, it is easy for the physician to confuse the wires at their proximal ends outside of the patient or within the guide.

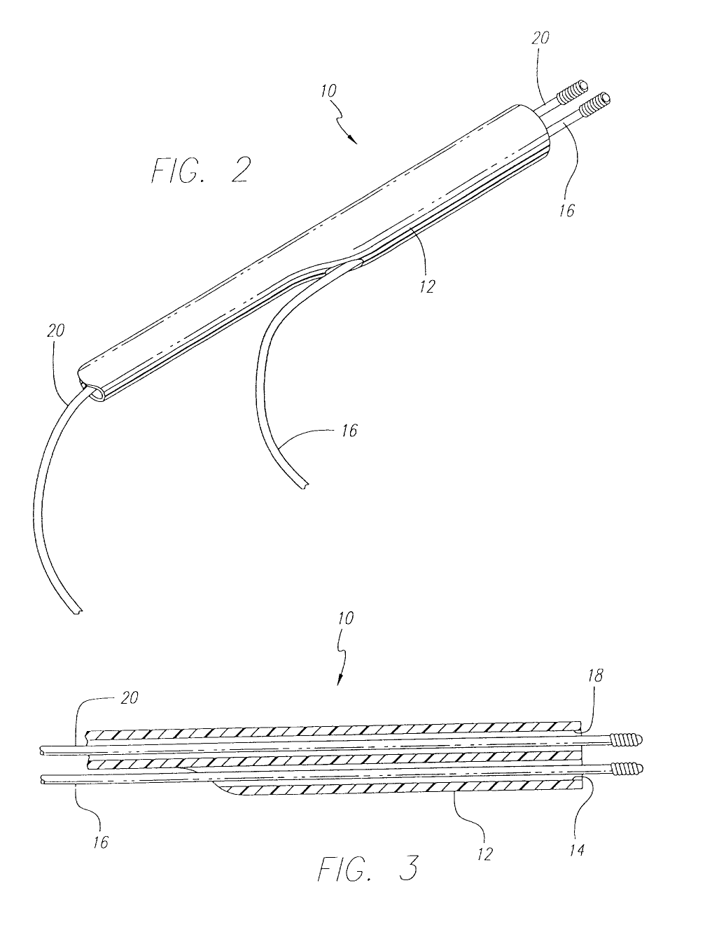

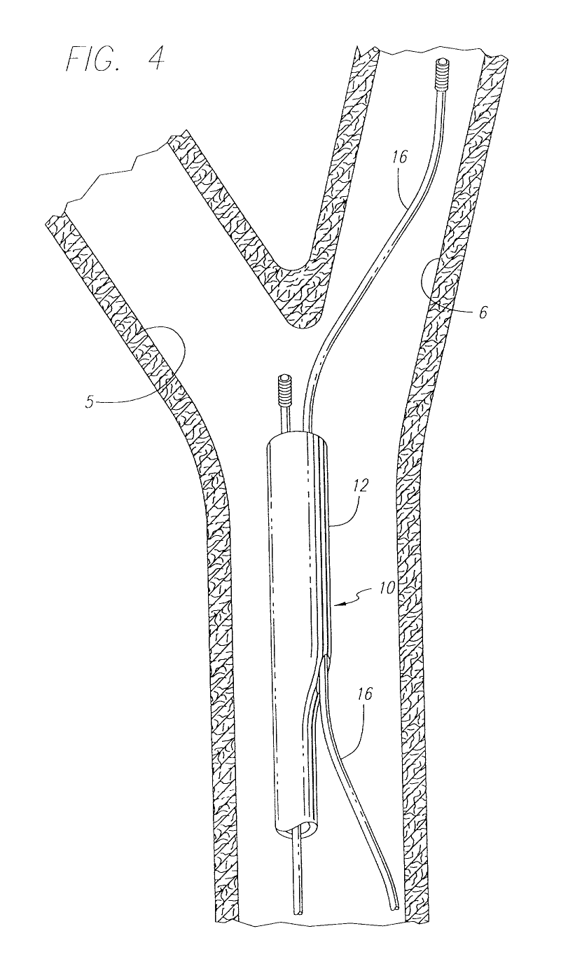

Turning to FIGS. 2 and 3, in one embodiment of the present invention, a delivery assembly 10 is provided for delivering guide wires to a bifurcated vessel in preparation of interventional treatment. A novel elongated catheter 12 has a proximal end to be manipulated by a physician from outside the body of a patient. The catheter further has a distal end that can be delivered into the patient by manipulation at the proximal end of the catheter. The catheter has two lumens disposed substantially parallel to each other therein. A tracking wire lumen 14 is configured for receiving a tracking guide wire 16. The tracking guide wire lumen extends through at least a portion of the catheter. The tracking guide wire lumen 14 is provided in a so-called rapid-exchange (RX) type system in this embodiment, which in one embodiment includes a short segment of tubing that receives the guide wire, rather than a long piece of tubing. Alternatively, the tubing can be long. The tracking guide wire can be unzipped through a slit in the side of the tubing. Rapid-exchange type catheters are well-known in the art. Alternatively, it is contemplated that the tracking guide wire lumen can be of the so-called unzippable-rapid-exchange type system, which includes a relatively short segment of tubing that does not include a slit. This type of system is also known to those skilled in the art. The tracking guide wire lumen can alternatively be of the so-called over-the-wire (OTW) type system, known in the art. This latter type of system is embodied in a long piece of tubing that extends throughout the length of the catheter.

An integrated guide wire lumen 18 is configured for receiving integrated guide wire 20. The integrated guide wire lumen extends through at least a portion of the catheter 12. The integrated guide wire lumen 18 is provided in an over-the-wire configuration and extends from the proximal end of the catheter through the distal end of the catheter in this embodiment. Alternatively, it is contemplated that the integrated guide wire lumen can be of the unzippable-rapid-exchange or rapid-exchange type. Moreover, the tracking guide wire lumen and the integrated guide wire lumen run substantially parallel to each other throughout their lengths. The tracking guide wire lumen and the integrated guide wire lumen do not move apart with respect to each other as the catheter is manipulated.

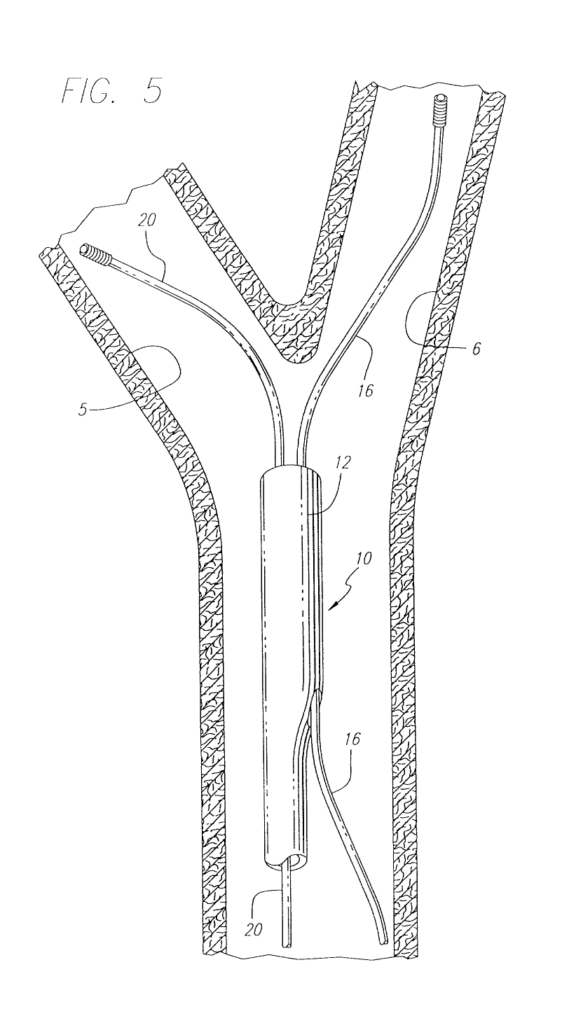

Referring now to FIGS. 4 and 5, in one method of use, after insertion of the distal end of tracking guide wire 16 into the patient, the physician manipulates the proximal end of the tracking guide wire from outside of the patient. The distal end of the tracking guide wire is advanced into main vessel 6 distal to the bifurcation with the proximal end of the tracking guide wire remaining outside the patient for the physician to further manipulate. This advancement of the tracking guide wire can be accomplished using conventional techniques including fluoroscopy or other imaging instrumentation.

Next, the proximal end of catheter 12 is pushed by the physician such that the distal end is advanced along tracking guide wire 16 which is positioned within tracking guide wire lumen 14. The catheter is advanced within main vessel 6 until the distal end of the catheter is just proximal to side branch vessel 5. It is contemplated that up to this point integrated guide wire 20 can reside in integrated guide wire lumen 18 so that the distal end of the integrated guide wire is near the distal end of the integrated guide wire lumen. Alternatively, the integrated guide wire 20 can now be inserted into the proximal end of the integrated guide wire lumen 18 and advanced through the catheter. The integrated guide wire 20 exits the distal end of the integrated guide wire lumen 18. The integrated guide wire 20 is then further advanced into the side branch vessel by means of the physician pushing the proximal end of the integrated guide wire 20 from outside the body.

In keeping with the invention, the catheter can now be withdrawn from the patient's vasculature in a known rapid-exchange manner or by other appropriate means. In other words, the wires can be pulled out through slits (not shown), or “unzipped,” from the catheter lumens if the lumens are of the rapid-exchange type. The catheter can also be withdrawn from the patient's vasculature using an over-the-wire technique, requiring exchange length wires. One of the two wires can be an exchange length wire and the other a standard length wire used in a rapid-exchange technique. The invention is suitable for any of these combinations. Thus, the withdrawal of the catheter is accomplished without wire wrapping or crossing due to the novel design of the present invention.

In keeping with the invention, FIG. 6 shows one possible design of a retaining element, such as a clip 30, that can be attached to tracking guide wire 16 and integrated guide wire 20 outside of the body of the patient. The slits 32, or other suitable means, in the clip 30 retain the guide wires, yet allow the guide wires to slide therethrough. Furthermore, the guide wires can be easily and quickly removed from the clip at any time. In one embodiment, the slits are cut into protrusions 34 and extend throughout the height thereof. One protrusion is located on each side of block 36. Lateral faces 38 can be tapered in one embodiment.

The clip 30 serves the important function of keeping the guide wires from crossing and wrapping outside the body. The clip 30 therefore helps the physician prevent making the common mistake of confusing the tracking guide wire with the integrated guide wire during an interventional procedure. The clip 30 can be made of rubber, plastic, or any other suitable material. The distance between slits 32 can vary as the application requires.

It is contemplated that the retaining element can assume other shapes adequate for retaining guide wires. Thus, two guide wires have been delivered to a bifurcation without the commonly associated problems of wire wrapping and wire crossing. The bifurcation is thus ready for appropriate interventional treatment such as the delivery of a bifurcated stent, which is known in the art.

As previously described, it is contemplated that tracking guide wire lumen 14 and integrated guide wire lumen 18 can be embodied in various ways including so-called over-the-wire, rapid-exchange, and unzippable-rapid-exchange.

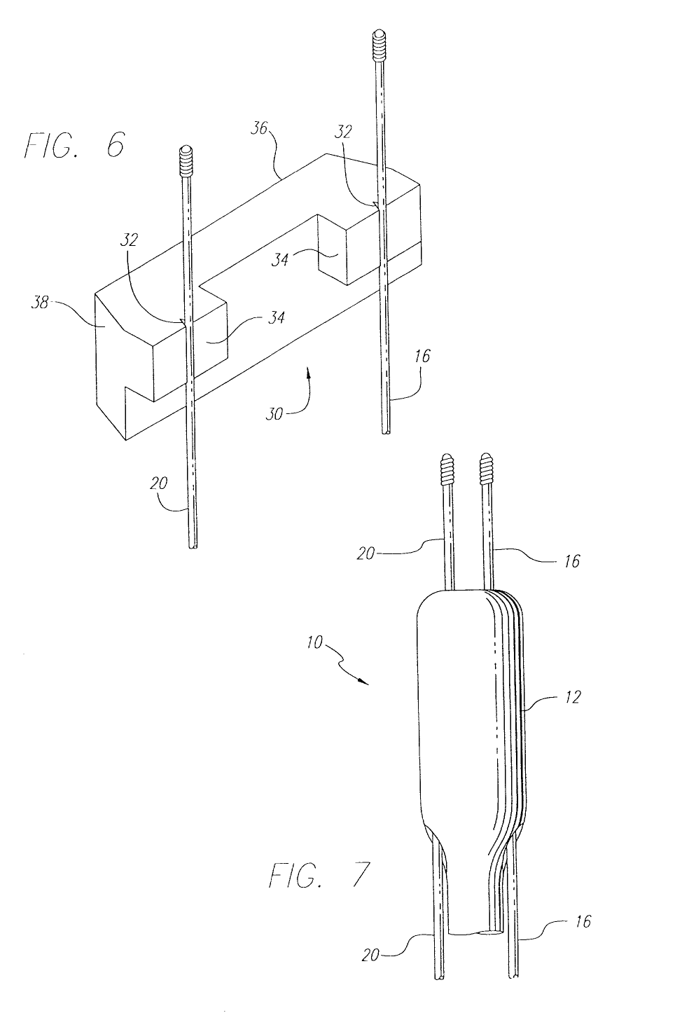

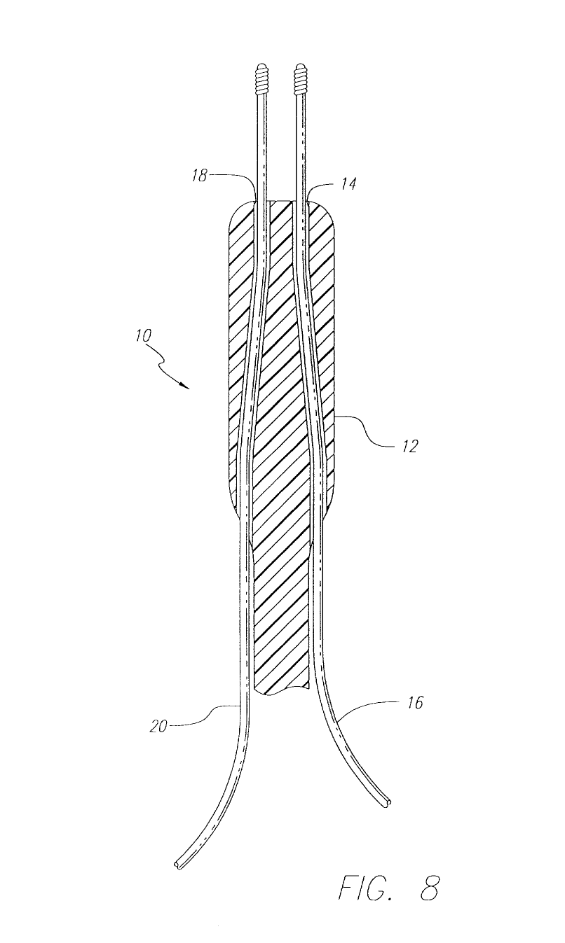

FIGS. 7 and 8 show another possible design of catheter 12 that can be used in conjunction with the methods of the present invention. In keeping with the invention, delivery assembly 10 can be embodied in elongated catheter 12 having two rapid-exchange (or unzippable-rapid-exchange) lumens 14, 18 disposed substantially parallel to each other therein. The catheter has a proximal end to be manipulated by a physician from outside the body of a patient. The catheter further has a distal end that can be delivered into the patient by manipulation at the proximal end of the catheter. A tracking wire lumen 14 is configured for receiving tracking guide wire 16. The tracking guide wire lumen extends through at least a portion of the catheter. An integrated guide wire lumen 18 is configured for receiving integrated guide wire 20. The integrated guide wire lumen extends through at least a portion of the catheter. The tracking guide wire lumen and the integrated guide wire lumen run substantially parallel to each other throughout their lengths. The tracking guide wire lumen and the integrated guide wire lumen do not move apart with respect to each other as the catheter is manipulated. Therefore, guide wires 16, 20 can be delivered to a bifurcation without the problems of wire wrapping and crossing.

While the invention herein has been illustrated and described in terms of a delivery catheter assembly for treating bifurcated vessels, it will be apparent to those skilled in the art that the invention can be used in other instances. Other modifications and improvements may be made without departing from the scope of the invention.

What is claimed:

1. A catheter assembly for treating bifurcated vessels having a main vessel and a side branch vessel, comprising:
an elongated catheter;
a tracking guide wire lumen for receiving a tracking guide wire, the tracking guide wire lumen extending through at least a portion of the catheter;
an integrated guide wire lumen for receiving an integrated guide wire, the integrated guide wire lumen extending through at least a portion of the catheter;
a tracking guide wire positionable within the tracking guide wire lumen;
an integrated guide wire positionable within the integrated guide wire lumen; and
a retaining element for retaining the tracking guide wire and the integrated guide wire.

2. The catheter assembly of claim 1, wherein the retaining element has a clip for retaining a guide wire.

3. The catheter assembly of claim 1, wherein the tracking guide wire lumen is of the over-the-wire type.

4. The catheter assembly of claim 1, wherein the tracking guide wire lumen is of the rapid exchange type.

5. The catheter assembly of claim 4, wherein the tracking guide wire lumen is unzippable.

6. The catheter assembly of claim 1, wherein the integrated guide wire lumen is of the over-the-wire type.

7. The catheter assembly of claim 1, wherein the integrated guide wire lumen is of the rapid exchange type.

8. The catheter assembly of claim 7, wherein the integrated guide wire lumen is unzippable.

9. The catheter assembly of claim 1, wherein the integrated guide wire lumen extends from a proximal end through a distal end of the catheter.

10. A method of preparing a bifurcated vessel having a bifurcation, a main vessel, and a side branch vessel, for an interventional procedure, comprising the steps of:
providing an elongated catheter;
providing a tracking guide wire and tracking guide wire lumen for receiving the tracking guide wire, the tracking guide wire lumen extending through at least a portion of the catheter;
providing an integrated guide wire and integrated guide wire lumen for receiving the integrated guide wire, the integrated guide wire lumen extending through at least a portion of the catheter;
wherein the tracking guide wire lumen and the integrated guide wire lumen run substantially parallel to each other throughout their lengths, and the tracking guide wire lumen and the integrated guide wire lumen do not move apart with respect to each other;
back loading the tracking guide wire into the tracking guide wire lumen;
advancing the catheter over the tracking guide wire to a position proximal of the bifurcation in the main vessel;
advancing the integrated guide wire through the integrated guide wire lumen and into the side vessel branch;
removing the catheter from a patient's vasculature;
providing a retaining element for retaining the tracking guide wire and the integrated guide wire; and
maintaining the position of the tracking guide wire relative to the integrated guide wire with the retaining element.
